(19) United States
(12) Patent Application Publication
(10) Pub. No.: US 2019/0060101 A1
(43) Pub. Date: Feb. 28, 2019

(54) LEG SUPPORT AND STABILIZING DEVICE

(71) Applicant: Adwait P. Muthal, Waltham, MA (US)

(72) Inventor: Adwait P. Muthal, Waltham, MA (US)

(21) Appl. No.: 16/110,650

(22) Filed: Aug. 23, 2018

Related U.S. Application Data
(60) Provisional application No. 62/552,804, filed on Aug. 31, 2017.

(51) Int. Cl. A61F 5/37; A61F 5/01

(52) U.S. Cl. CPC A61F 5/3715; A61F 5/0104

(57) ABSTRACT

The present invention relates to a device suitable for supporting and stabilizing the legs of a user during long trips. In particular, the present invention relates to a device including a dual leg support contoured block to be placed between the legs of a user and straps to be wrapped around the contoured block and the legs of a user to hold the legs in place throughout a duration of the trip.

### CROSS-REFERENCE TO RELATED APPLICATION(S)

This application claims priority to, and the benefit of, co-pending U.S. Provisional Application No. 62/552,804, filed Aug. 31, 2017, for all subject matter common to both applications. The disclosure of said provisional application is hereby incorporated by reference in its entirety.

### FIELD OF THE INVENTION

The present invention relates to a device suitable for supporting and stabilizing the legs of a user during long trips. In particular, the present invention relates to a device including a dual leg support contoured block to be placed between the legs of a user and straps to be wrapped around the contoured block and the legs of a user to hold the legs in place throughout a duration of the trip.

### BACKGROUND

Generally, when traveling on long trips, individuals sit in an upright position in a chair or seat. While seated in this position, it is common for individuals to attempt to sleep for some duration of the trip. While an individual is sleeping, or even while awake, it is common for their legs to move back and forth uncontrollably, or otherwise be positioned in configurations that place strain on knees or ankles of the individual. Movement of the legs of the individual can cause them to experience discomfort during and after the trip. In particular, the individual can experience pain in the lower back and knee regions. Similarly, the movement can cause the knees/legs of the individual to collide with one another and/or with nearby passengers, which can cause further discomfort and sleep disruption.

### SUMMARY

There is a need for a device to provide support and stabilization to the legs of individuals while sitting upright, such as would be suitable for use on long trips. The present invention is directed toward further solutions to address this need, in addition to having other desirable characteristics. Specifically, the present invention is directed to a device and system for supporting and stabilizing the legs of a user, such as while seated in an upright position while traveling. The device and system includes two main components that are designed to hold the user's knees and legs in place while keeping a predetermined amount of space between the knees and legs. The two main components include a dual leg support contoured block and one or more adjustable harness straps. Together these two components hold the knees and legs together in a specific position that provides improved stability while sitting on chair/seat and reduces undesirable strain on the user's knees, back, and ankles. As a result, the user is able to sleep more comfortably throughout the duration of a trip and reduce discomfort and pain caused by legs being in positions that cause undue strain on muscles and joints for extended periods of time.

In accordance with example embodiments of the present invention, leg support and stabilization device is provided. The device includes a dual leg support contoured block. The dual leg support contoured block includes a first concave leg receiving support on a first side of the contoured block and a second concave leg receiving support on a second side of the contoured block, opposite the first side. The device also includes a first adjustable harness strap coupled with the contoured block and sized, dimensioned, and adapted to wrap around a first leg of the user when the first leg is placed into the first concave leg receiving support, wherein the first adjustable harness strap removably attaches to the contoured block, thereby stabilizing the first leg in place against the first concave leg receiving support, The device further includes a second adjustable harness strap coupled with the contoured block and sized, dimensioned, and adapted to wrap around a second leg of the user when the second leg is placed into the second concave leg receiving support, wherein the second adjustable harness strap removably attaches to the contoured block thereby stabilizing the second leg in place against the second concave leg receiving support. The first concave leg receiving support and the second concave leg receiving support are adapted to support the legs of the user and the first adjustable harness strap and the second adjustable harness strap are adapted to stabilize the first leg and the second leg of the user in place against the contoured block, thereby reducing strain on the legs of the user when in a seated position. Additionally, the contoured block and adjustable harness straps are not required to be affixed or attached to a seat for operation.

In accordance with aspects of the present invention, the first adjustable harness strap and the second adjustable harness strap further comprises a first end and a second end. The first end of the first adjustable harness strap and the second end the second adjustable harness strap can couple together on the top of the contoured block and the first leg and the second leg of the user to form an elliptical loop shape.

In accordance with aspects of the present invention, the tightening of adjustable straps of the first adjustable harness strap and the second adjustable harness strap secures the thighs of the user against the first concave leg receiving support and the second concave leg receiving support of the contoured block and stabilizes the first leg and the second leg of the user. The contoured block can maintain a distance between the first leg and the second leg of the user such that knees of the user do not contact one another. The first adjustable harness strap and the second adjustable harness strap can include hook and loop fasteners attaching means. The first adjustable harness strap and the second adjustable harness strap can be constructed from at least one of cloth, canvas, and elastic material. The first adjustable harness strap and the second adjustable harness strap can be removably attached to the contoured ort block. The first adjustable harness strap and the second adjustable harness strap can be fixedly attached to the contoured block.

In accordance with example embodiments of the present invention, a leg support and stabilization device is provided. The device includes a dual leg support contoured block. The dual leg support contoured block includes a first concave leg receiving support on a first side of the contoured block and a second concave leg receiving support on a second side of the contoured block, opposite the first side and adapted to wrap around a second leg of the user when the second leg is placed into the second concave leg receiving support. The device also includes an adjustable harness strap coupled with the contoured block and sized, dimensioned, and adapted to wrap around a first leg of the user when the first leg is placed into the first concave leg receiving support. The adjustable harness strap removably attaches to the contoured block, thereby stabilizing the first leg in place against the first concave leg receiving support. The first concave leg receiving support and the second concave leg receiving support are adapted to support the legs of the user and the adjustable harness strap is adapted to stabilize the first leg and the second leg of the user in place against the contoured block, thereby reducing strain on the legs of the user when in a seated position. The contoured block and adjustable harness straps are not required to be affixed or attached to a seat for operation.

In accordance with example embodiments of the present invention, a leg support and stabilization device as described herein and all operable equivalents and variations thereof is provided.

In accordance with example embodiments of the present invention, method for using a leg support and stabilization device as described herein and all operable equivalents and variations thereof is provided.

### BRIEF DESCRIPTION OF THE FIGURES

These and other characteristics of the present invention will be more fully understood by reference to the following detailed description in conjunction with the attached drawings, in which:

FIG. 1 is an illustrative front view of a leg support and stabilization device, in accordance with the present invention;

FIG. 2 is an illustrative example of the leg support and stabilization device in use;

FIG. 3 is a perspective view of the leg support and stabilization device;

FIG. 4 is a right side view of the leg support and stabilization device;

FIG. 5 is a left side view of the leg support and stabilization device; and

FIG. 6 is a flowchart depicting an example method of use of the leg support and stabilization device in accordance with the present invention.

### DETAILED DESCRIPTION

An illustrative embodiment of the present invention relates to a leg support and stabilization device suitable for use during long trips in which the user is seated upright for a long period of time. The device includes a dual leg support contoured block configured to rest on a seat between the legs of the user. The leg support is shaped to conform to the shape of the legs of the user to provide comfortable support and fixed separation between the legs. The device also includes an adjustable harness strap or straps configured to wrap under and around the leg support and legs of the user. The adjustable harness provides a means to secure the legs in place adjacent with the contoured block such that the legs of the user do not separate beyond the area defined by the harness. The combination of the contoured block and the harness provides a means to comfortably maintain a stable positioning of the legs of a user while in a seated position, suitable for traveling or other extended durations. In particular, the contoured block keeps the legs separated by a predetermined distance while preventing the legs from knocking into one another, while the harness prevents the legs from expanding outward beyond the separation created by the contoured block and perhaps colliding with surrounding objects. The stabilization provided by the device creates consistent positioning of the legs of a user in a way that prevents unintended movement of the legs (e.g., while sleeping) that will cause the user back or knee pain.

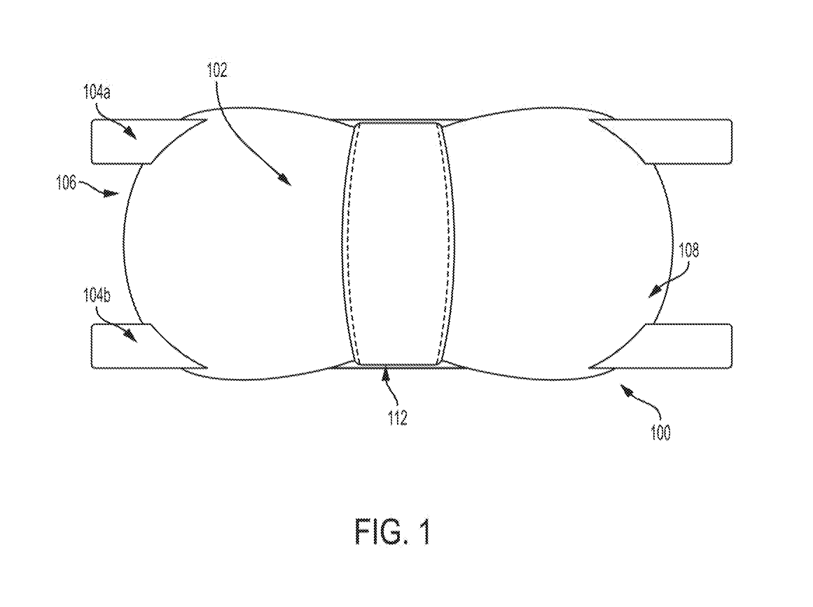

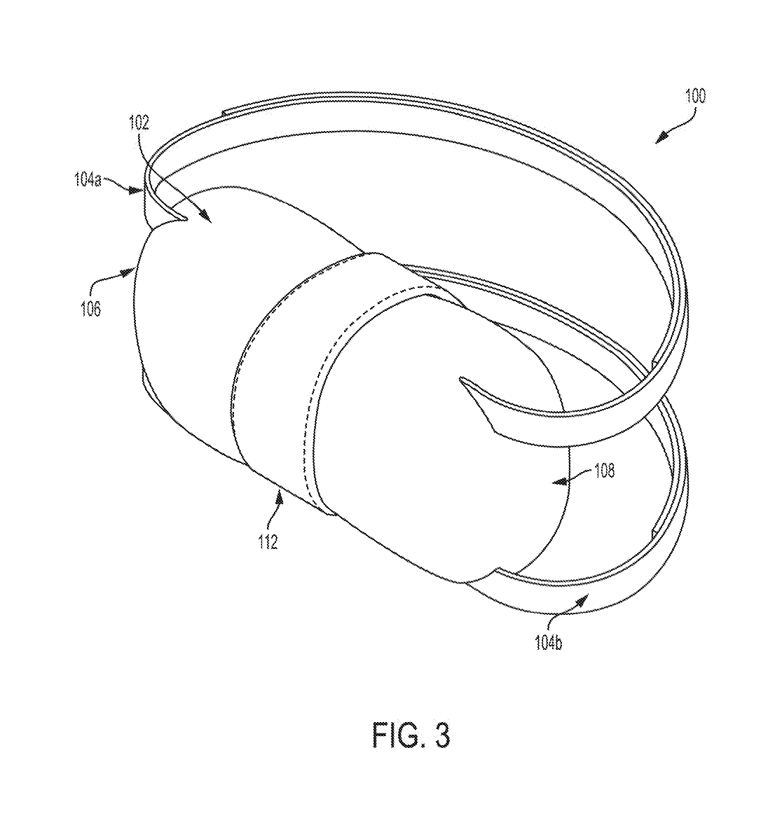

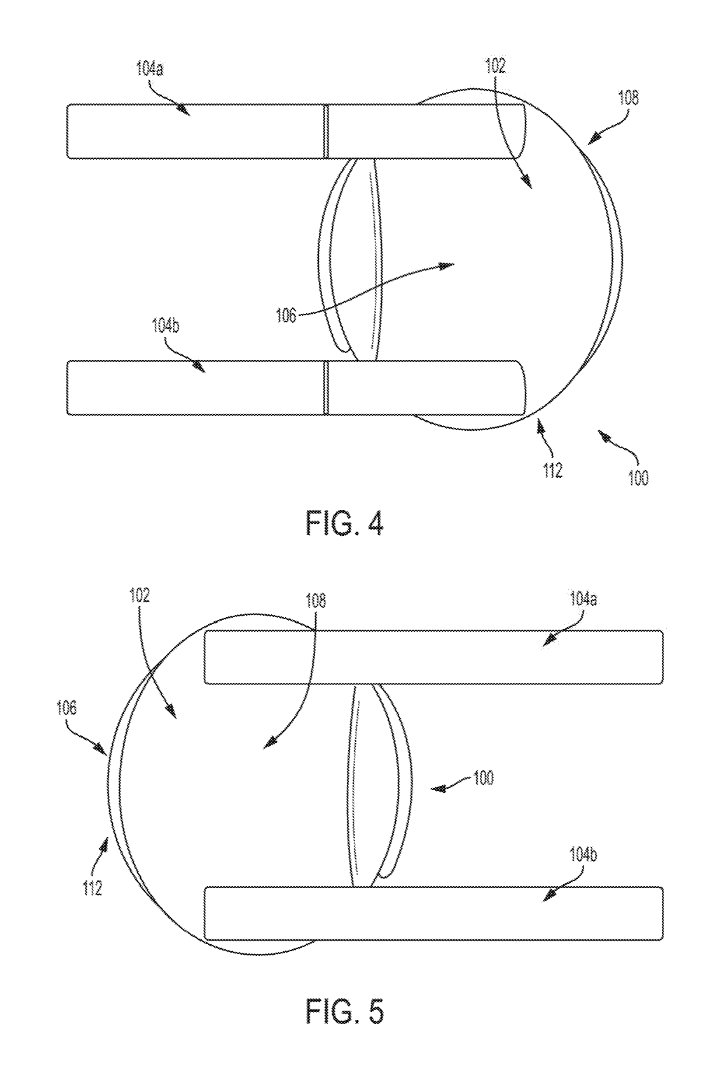

FIGS. 1 through 6, wherein like parts are designated by like reference numerals throughout, illustrate an example embodiment or embodiments of a leg support and stabilization device, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIGS. 1, 3 and 4 depict an illustrative example of a leg support and stabilization device 100 in accordance with the present invention. In accordance with an example embodiment of the present invention, the device 100 includes a dual leg support contoured block 102 and one or more adjustable harness straps 104. The contoured block 102 includes a first concave leg receiving support 106 on a first side of the contoured block 102 and a second concave leg receiving support 108 on a second side of the contoured block 102. The second concave leg receiving support 108 is positioned opposite the first concave leg receiving support 106 forming a curved elongate X shape, as depicted in FIG. 1. As would be appreciated by one skilled in the art, the contoured block 102 can be formed from any suitable material that is capable of maintaining the dual concave shape of the leg receiving supports 106, 108. For example, the contoured block 102 can be formed from a soft foam material, hard form, elastic memory foam, recyclable shredded paper, dried beans, polyvinyl chloride pellets, closed-cell extruded polystyrene foam, etc.

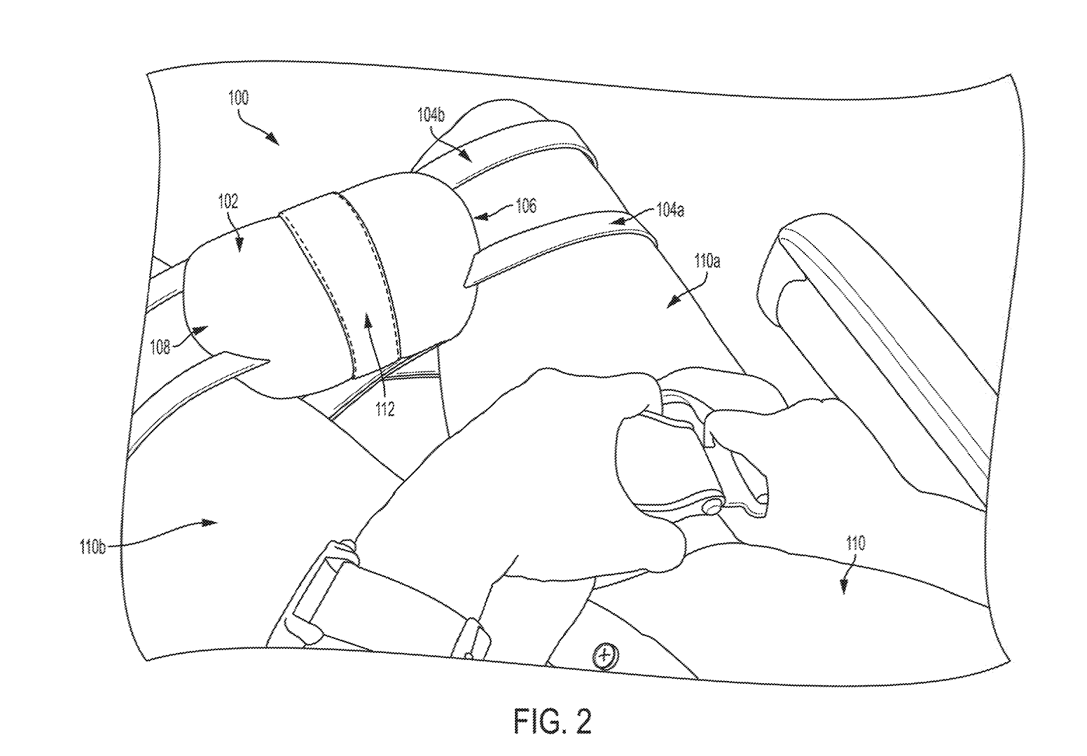

Additionally, the contoured block 102 and the leg receiving supports 106, 108 are shaped, sized and dimensioned to comfortably support and separate legs of a user 110 (see FIG. 2) when the contoured block 102 is placed between the legs of the user 110. For example, the contoured block 102 can be about 4 inches to about 6 inches wide, depending on a size of a user 110. The leg receiving supports 106, 108 of the contoured block 102 maintain a distance between the legs of the user 110 such that knees of the user 110 do not contact one another. The contoured block 102 can include a variety of size and shape combinations without departing from the scope of the present invention. For example, the contoured block 102 can include a cylindrical, triangular, rectangular, or other polygonal shape. Similarly, the contoured block 102 can be formed from a plurality of shapes coupled together (e.g., by the harness strap, hook and loop fastener, or the like) to form a single leg separation means.

Continuing with FIGS. 1, 3, and 4, in accordance with an example embodiment of the present invention, the device 100 includes a first adjustable harness strap 104a and a second adjustable harness strap 104b individually coupled with the contoured block 102. As would be appreciated by one skilled in the art, the adjustable harness strap 104 can include a single strap or multiple harness straps combined to act as a single harness strap. The adjustable harness straps 104a, 104b can be attached to the contoured block 102 through any combination of systems and methods known in the art. For example, the adjustable harness straps 104a, 104b can be removably attached to the contoured block 102 via a combination of hook and loop fastener, buttons, snaps, ties, or other known fastener means. As would be appreciated by one skilled in the art, the adjustable harness straps 104a, 104b can also be fixedly attached to the contoured block 102. However, the contoured block 102 and adjustable harness straps 104a, 104b are not required to be affixed or attached to a seat for operation.

Additionally, (returning to FIG. 2) the first adjustable harness strap 104a is sized, dimensioned, and adapted to wrap around a first leg 110a of the user 110 when the first leg 110a is placed into/adjacent to the first concave leg receiving support 106 of the contoured block 102. Similarly, the second adjustable harness strap 104b is sized, dimensioned, and adapted to wrap around a second leg 110b of the user 110 when the second leg 110b is placed into/adjacent to the second concave leg receiving support 108 of the contoured block 102. The first adjustable harness strap 104a and the second adjustable harness strap 104b are adapted to stabilize the first leg 110a and the second leg 110b of the user 110 in place against the contoured block 102, thereby reducing strain on the legs of the user 110 when in a seated position. In particular, tightening of the adjustable harness straps 104a, 104b secures the thighs of the user 110 against the first concave leg receiving support 106 and the second concave leg receiving support 108 of the contoured block 102 and stabilizes the first leg 110a and the second leg 110b of the user 110 respectively.

In accordance with an example embodiment of the present invention, the first adjustable harness strap 104a and the second adjustable harness strap 104b each include a first end and a second end. Additionally, the first end of the first adjustable harness strap 104a and the second end the second adjustable harness strap 104b are coupled together on the top of the contoured block 102 to form an elliptical loop shape around the legs 110a, 110b of the user 110. As would be appreciated by one skilled in the art, the first end of the first adjustable harness strap 104a and the second end the second adjustable harness strap 104b can couple together using any means known in the art. For example, the first adjustable harness strap 104a and the second adjustable harness strap 104b can include hook and loop fastener, buttons, snaps, ties, or other known attaching means. Regardless of implementation, each of the adjustable harness straps 104a, 104b are individually adjustable to fit legs of different sizes and shapes. Additionally, the first adjustable harness strap 104a and the second adjustable harness strap 104b can be constructed from at least one of cloth, canvas, and elastic material.

In accordance with an example embodiment of the present invention, the first adjustable harness strap 104a and the second adjustable harness strap 104b are designed to individually wrap around both legs 110a, 110b of a user 110. FIG. 2 depicts an illustrative example in which the first adjustable harness strap 104a and the second adjustable harness strap 104b individually wrap around both legs 110a, 110b of the user. In the example embodiment of FIG. 2, the first adjustable harness strap 104a is wrapped around both legs 110a, 110b above a knee of the user 110 and the second adjustable harness strap 104b is wrapped around both legs 110a, 110b below a knee of the user 110. Additionally, as depicted in FIG. 2, the device 100 include brace straps 112 that tether the adjustable harness straps 104a, 104b together and the adjustable harness straps 104a, 104b with the contoured block 102. The adjustable harness straps 104a, 104b are connected to the contoured block 102 so that the contoured block 102 is held between the knees and the adjustable harness straps 104a, 104b go around the knees to gently tighten the knees together.

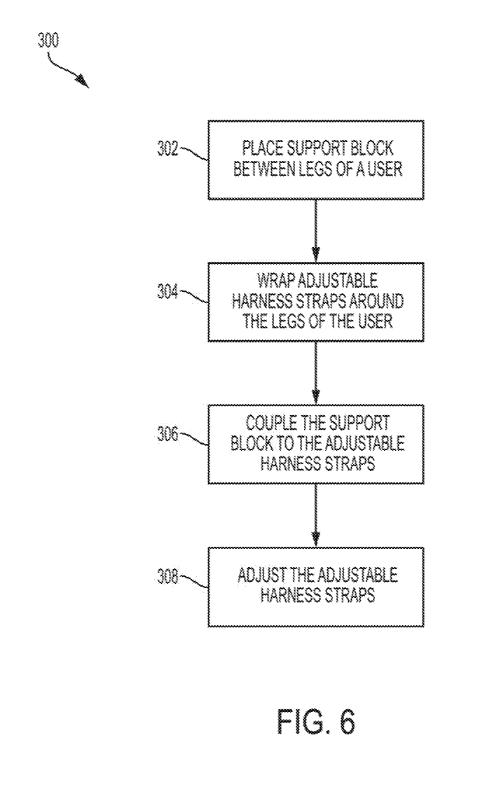

FIG. 6 depicts an example method 300 of use for the support and stabilization device in accordance with the present invention. Initially, a user 110 places the contoured block 102 between their legs 110a, 110b while seated (STEP 302). As would be appreciated by one skilled in the art, the contoured block 102 can be place at any location between the legs 110a, 110b of the user 110. For example, the contoured block 102 can be placed between the thighs, knees, or shins of the user 110. Thereafter, the adjustable harness straps 104a, 104b are wrapped under and around the legs 110a, 110b of the user 110, depending on the strap design, and coupled together on a lap of the user 110 (STEP 304). As would be appreciated by one skilled in the art, based on the harness design the straps will be wrapped around different locations of the user's legs 110a, 110b.

Prior or subsequent to placing the contoured block 102 and the adjustable harness straps 104a, 104b, the user 110 couples the adjustable harness straps 104a, 104b to the contoured block 102 (STEP 306). Once the adjustable harness straps 104a, 104b are wrapped around the legs 110a, 110b of the user 110, the user 110 adjusts and tightens the adjustable harness straps 104a, 104b such that the inner legs/thighs of the user 110 are held comfortably against the concave shape of the leg receiving supports 106, 108 of the contoured block 102 (STEP 308).

In operation, the contoured block 102 prevents the legs 110a, 110b from moving closer together and colliding while the adjustable harness straps 104a, 104b prevent the legs 110a, 110b from separating further than the secured position (e.g., adjacent to the contoured block 102). The contoured block 102 and the adjustable harness straps 104a, 104b in combination provide the support and stabilization to the legs 110a, 110b of the user such that the legs 110a, 110b do not move freely while the user 110 sits/sleeps throughout a trip or other extended duration of sitting. The support and stabilization provided by the device 100 help to reduce pain felt after a long trip in which a user is sitting upright. In particular, the stabilized distance between the legs 110a, 110b and pressure applied by the adjustable harness straps 104a, 104b helps to keep muscles of legs activated and relieves pressure on lower back and impact due to sudden movements during inactivity/sleep. Additionally, the support and stabilization enable the user to sleep comfortable because the legs are not moving freely while asleep.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A leg support and stabilization device, the device comprising:
a dual leg support contoured block, comprising
a first concave leg receiving support on a first side of the contoured block;
a second concave leg receiving support on a second side of the contoured block, opposite the first side;
a first adjustable harness strap coupled with the contoured block and sized, dimensioned, and adapted to wrap around a first leg of the user when the first leg is placed into the first concave leg receiving support, wherein the first adjustable harness strap removably attaches to the contoured block, thereby stabilizing the first leg in place against the first concave leg receiving support;
a second adjustable harness strap coupled with the contoured block and sized, dimensioned, and adapted to wrap around a second leg of the user when the second leg is placed into the second concave leg receiving support, wherein the second adjustable harness strap removably attaches to the contoured block thereby stabilizing the second leg in place against the second concave leg receiving support;
wherein the first concave leg receiving support and the second concave leg receiving support are adapted to support the legs of the user and the first adjustable harness strap and the second adjustable harness strap are adapted to stabilize the first leg and the second leg of the user in place against the contoured block, thereby reducing strain on the legs of the user when in a seated position; and
wherein the contoured block and adjustable harness straps are not required to be affixed or attached to a seat for operation.

2. The device of claim 1, wherein the first adjustable harness strap and the second adjustable harness strap further comprises a first end and a second end.

3. The device of claim 2, wherein the first end of the first adjustable harness strap and the second end the second adjustable harness strap couple together on the top of the contoured block and the first leg and the second leg of the user to form an elliptical loop shape.

4. The device of claim 1, wherein tightening of adjustable straps of the first adjustable harness strap and the second adjustable harness strap secures the thighs of the user against the first concave leg receiving support and the second concave leg receiving support of the contoured block and stabilizes the first leg and the second leg of the user.

5. The device of claim 1, wherein the contoured block maintains a distance between the first leg and the second leg of the user such that knees of the user do not contact one another.

6. The device of claim 1, wherein the first adjustable harness strap and the second adjustable harness strap include hook and loop fasteners attaching means.

7. The device of claim 1, wherein the first adjustable harness strap and the second adjustable harness strap are constructed from at least one of cloth, canvas, and elastic material.

8. The device of claim 1, wherein the first adjustable harness strap and the second adjustable harness strap are removably attached to the contoured ort block.

9. The device of claim 1, wherein the first adjustable harness strap and the second adjustable harness strap are fixedly attached to the contoured block.

10. A leg support and stabilization device, the device comprising:
a dual leg support contoured block, comprising
a first concave leg receiving support on a first side of the contoured block;
a second concave leg receiving support on a second side of the contoured block, opposite the first side and adapted to wrap around a second leg of the user when the second leg is placed into the second concave leg receiving support;
an adjustable harness strap coupled with the contoured block and sized, dimensioned, and adapted to wrap around a first leg of the user when the first leg is placed into the first concave leg receiving support,
wherein the adjustable harness strap removably attaches to the contoured block, thereby stabilizing the first leg in place against the first concave leg receiving support;
wherein the first concave leg receiving support and the second concave leg receiving support are adapted to support the legs of the user and the adjustable harness strap is adapted to stabilize the first leg and the second leg of the user in place against the contoured block, thereby reducing strain on the legs of the user when in a seated position; and
wherein the contoured block and adjustable harness straps are not required to be affixed or attached to a seat for operation.

11. A method for using a leg support and stabilization device; comprising:
the leg support and stabilization device comprising:
a dual leg support contoured block, comprising a first concave leg receiving support on a first side of the contoured block, and a second concave leg receiving support on a second side of the contoured block, opposite the first side;
a first adjustable harness strap coupled with the contoured block and sized, dimensioned, and adapted to wrap around a first leg of the user when the first leg is placed into the first concave leg receiving support, wherein the first adjustable harness strap removably attaches to the contoured block, thereby stabilizing the first leg in place against the first concave leg receiving support; and
a second adjustable harness strap coupled with the contoured block and sized, dimensioned, and adapted to wrap around a second leg of the user when the second leg is placed into the second concave leg receiving support, wherein the second adjustable harness strap removably attaches to the contoured block thereby stabilizing the second leg in place against the second concave leg receiving support;
the method comprising:
placing the dual leg support contoured block between legs of the user;
wrapping the first adjustable harness strap around the first leg of the user;
wrapping the second adjustable harness strap around the second leg of the user;
coupling the dual leg support contoured block and the first and second adjustable harness straps; and
adjusting the adjustable harness straps to adjust fit to the user.
